(19) United States
(12) Patent Application Publication
(10) Pub. No.: US 2026/0174669 A1
(43) Pub. Date: Jun. 25, 2026

(54) Composition for inhibiting generation of melanin and the use thereof

(71) Applicants: Dao Lung Steven Lin, New Taipei City (TW); Spirit Scientific Co., Ltd., New Taipei City (TW)

(72) Inventors: DAO LUNG STEVEN LIN, New Taipei City (TW); Chin Ho Chen, New Taipei City (TW)

(21) Appl. No.: 19/411,845

(22) Filed: Dec. 8, 2025

Related U.S. Application Data
(60) Provisional application No. 63/736,121, filed on Dec. 19, 2024.

(51) Int. Cl. A61K 8/98; A61K 8/02; A61K 8/64; A61Q 19/02

(52) U.S. Cl. CPC A61K 8/983; A61K 8/022; A61K 8/64; A61Q 19/02; A61K 2800/84

(57) ABSTRACT

A composition for inhibiting generation of melanin and the use thereof is provided, the composition includes platelets and/or growth factors isolated from blood, the composition for inhibiting generation of melanin has the abilities for inhibiting activity of tyrosinase and alleviating inflammation, and the composition for inhibiting generation of melanin is able to be used to manufacture of preparation for improving skin pigmentation or alleviating melasma.

### CROSS REFERENCE TO RELATED APPLICATION

This application claims the benefit of U.S. Provisional Patent Application No. 63/736,121 filed on Dec. 19, 2024, which is incorporated by reference herein in its entirety.

### FIELD OF THE INVENTION

The present invention relates to a composition for inhibiting generation of melanin and the use thereof, in particular, to a composition comprising platelets and/or growth factors isolated from blood for inhibiting generation of melanin and the use for manufacturing of a preparation for inhibiting generation of melanin in a subject.

### BACKGROUND OF THE INVENTION

Skin is the largest organ in the human body and is divided into epidermis, which is composed of stratum corneum and basal layer; dermis and subcutaneous tissue. The color of skin is affected by a variety of pigments, including melanin, carotene, and hemoglobin. Melanin is produced by melanocytes, which are mainly distributed in the stratum basale.

Tyrosinase is an oxidase and a rate-limiting enzyme that regulates generation of melanin. When melanocytes are stimulated, they activate the activity of tyrosinase, which initiates a series of chemical reactions and finally produces melanin. When melanosomes containing a large amount of melanin are released into keratinocytes, they cause the skin to darken or form various spots (e.g., nevus, melasma). Therefore, inhibiting the production and activity of tyrosinase is considered a key step in reducing melanin generation, but when melanin is not produced, it causes vitiligo and leads to additional skin conditions.

Currently, commonly used whitening ingredients on the market are mainly added to cosmetics or skin care products to inhibit melanin formation and prevent pigment spots. If it is not added according to the concentration limit, it may cause allergies, apoptosis or death of melanocytes. Therefore, there is still a need for a composition or formulation that can reduce melanin generation without causing melanocyte apoptosis or death.

### BRIEF SUMMARY OF THE INVENTION

The present invention is to provide a composition for inhibiting generation of melanin, which comprises platelets and/or growth factors isolated from blood.

The composition comprises platelets with the amounts of 2.5×10⁷˜10×10⁹ platelets; Preferably, the composition comprises platelets with the amounts of 2.5×10⁷˜10×10⁸ platelets; Most preferred, the composition comprises platelets with the amounts of 5×10⁷˜10×10⁸ platelets.

The composition comprises growth factors of platelet-derived growth factor-BB (PDGF-BB) and/or transforming growth factor (TGF-β).

The composition comprises PDGF-BB with a concentration of 0.1 ng/ml˜5 ng/ml; In a preferred manner, the composition comprises PDGF-BB with a concentration of 0.1 ng/ml˜3 ng/ml; In another preferred manner, the composition comprises PDGF-BB with a concentration of 0.25 ng/ml˜2 ng/ml; Most preferred, the composition comprises PDGF-BB with a concentration of 0.25 ng/ml˜1 ng/mL.

The composition comprises TGF-β with a concentration of 0.5 ng/ml˜50 ng/ml; In a preferred manner, the composition comprises TGF-β with a concentration of 0.5 ng/ml˜30 ng/ml; In another preferred manner, the composition comprises TGF-β with a concentration of 1 ng/mL˜20 ng/mL; Most preferred, the composition comprises TGF-β with a concentration of 1 ng/ml˜10 ng/mL.

In an embodiment, the composition is freeze-dried to form a dry powder, and the dry powder of the composition comprises 4×10⁸˜1.5×10¹¹ platelets per gram; In a preferred manner, the dry powder of the composition comprises 4×10⁸˜1.5×10¹⁰ platelets per gram; the dry powder of the composition comprises 8×10⁸˜1.5×10¹⁰ platelets per gram.

The present invention relates to a method for alleviating skin pigmentation by administering a composition to a subject suffering from skin pigmentation.

### BRIEF DESCRIPTION OF DRAWINGS

FIG. 1 shows result of the effect of different concentration of platelet-derived growth factor-BB (PDGF-BB) in the composition of the present invention on melanocyte proliferation.

FIG. 2 shows result of the effect of different concentration of platelet-derived growth factor-BB (PDGF-BB) in the composition of the present invention on melanin generation.

FIG. 3 shows result of different concentration of platelet-derived growth factor-BB (PDGF-BB) in the composition of the present invention inhibiting the generation of melanin.

FIG. 4 shows result of different concentration of transforming growth factor (TGF-β) in the composition of the present invention inhibiting the generation of melanin.

FIG. 5 shows result of different amounts of platelets in the composition of the present invention inhibiting the generation of melanin.

FIG. 6 shows result of different amounts of platelets in the composition of the present invention and different whitening ingredients inhibiting inflammation of melanocytes.

FIG. 7 shows result of different amounts of platelets in the composition of the present invention and different whitening ingredients inhibiting the activity of tyrosinase.

### DETAIL DESCRIPTION OF THE INVENTION

The purpose of the present invention is to provide a composition for inhibiting generation of melanin, which comprises platelets and/or growth factors isolated from blood.

In one embodiment, the composition comprises platelets or growth factors isolated from blood, preferably, the composition comprises platelets isolated from plasma.

The composition preferably comprises 2.5×10⁷˜10×10⁹ platelets.

The composition preferably comprises 2.5×10⁷˜10×10⁸ platelets.

The composition preferably comprises 5×10⁷˜10×10⁸ platelets.

In another embodiment, the composition comprises platelet-derived growth factor-BB (PDGF-BB) and/or transforming growth factor (TGF-β).

In a preferred manner, the composition comprises PDGF-BB with a concentration of 0.1 ng/ml˜5 ng/mL.

In a preferred manner, the composition comprises PDGF-BB with a concentration of 0.1 ng/ml˜3 ng/mL.

In a preferred manner, the composition comprises PDGF-BB with a concentration of 0.25 ng/ml˜2 ng/mL.

In a preferred manner, the composition comprises PDGF-BB with a concentration of 0.25 ng/ml˜1 ng/mL.

In a preferred manner, the composition comprises TGF-β with a concentration of 0.5 ng/ml˜50 ng/mL.

In a preferred manner, the composition comprises TGF-β with a concentration of 0.5 ng/ml˜30 ng/mL.

In a preferred manner, the composition comprises TGF-β with a concentration of 1 ng/ml˜20 ng/ml.

In a preferred manner, the composition comprises TGF-β with a concentration of 1 ng/ml˜10 ng/ml.

In another embodiment, the composition for inhibiting generation of melanin used in the present invention is freeze-dried to form a dry powder.

In a preferred manner, the dry powder of the composition comprises 4×10⁸˜1.5×10¹¹ platelets per gram.

In a preferred manner, the dry powder of the composition comprises 4×10⁸˜1.5×10¹⁰ platelets per gram.

In a preferred manner, the dry powder of the composition comprises 8×10⁸˜1.5×10¹⁰ platelets per gram.

In a preferred manner, the dry powder of the composition comprises PDGF-BB with a concentration of 15˜850 ng per gram.

In a preferred manner, the dry powder of the composition comprises PDGF-BB with a concentration of 15˜500 ng per gram.

In a preferred manner, the dry powder of the composition comprises PDGF-BB with a concentration of 35˜350 ng per gram.

In a preferred manner, the dry powder of the composition comprises PDGF-BB with a concentration of 35˜200 ng per gram.

In a preferred manner, the dry powder of the composition comprises TGF-β with a concentration of 75˜8,500 ng per gram.

In a preferred manner, the dry powder of the composition comprises TGF-β with a concentration of 75˜5,000 ng per gram.

In a preferred manner, the dry powder of the composition comprises TGF-β with a concentration of 150˜3,500 ng per gram.

In a preferred manner, the dry powder of the composition comprises TGF-β with a concentration of 150˜2,000 ng per gram.

The another purpose of the present invention is to provide a method for alleviating skin pigmentation by administering a composition to a subject suffering from skin pigmentation.

In an embodiment, the pigmentation is caused by inflammation of cells.

In an embodiment, the pigmentation is induced by tyrosinase.

The another purpose of the present invention is to provide a method for alleviating melasma in a subject suffering from melasma by administering a composition to the subject suffering from melasma.

The composition for inhibiting generation of melanin in the present invention comprises 1×10⁷, 1.25×10⁷, 1.5×10⁷, 1.75×10⁷, 2×10⁷, 2.25×10⁷, 2.5×10⁷, 2.75×10⁷, 3×10⁷, 3.25×10⁷, 3.5×10⁷, 3.75×10⁷, 4×10⁷, 4.25×10⁷, 4.5×10⁷, 4.75×10⁷, 5×10⁷, 5.25×10⁷, 5.5×10⁷, 5.75×10⁷, 6×10⁷, 6.25×10⁷, 6.5×10⁷, 6.75×10⁷, 7×10⁷, 7.25×10⁷, 7.5×10⁷, 7.75×10⁷, 8×10⁷, 8.25×10⁷, 8.5×10⁷, 8.75×10⁷, 9×10⁷, 9.25×10⁷, 9.5×10⁷, 9.75×10⁷, 1×10⁸, 1.25×10⁸, 1.5×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰ platelets.

In the present invention, the composition for inhibiting generation of melanin comprises a growth factor isolated from blood, the growth factor is obtained by centrifuging whole blood samples, such as whole blood from non-human mammals or human, to obtain a high-concentration platelet-rich plasma (PRP), activators such as calcium chloride (CaCl₂)), thrombin, collagen, adenosine diphosphate (ADP), etc. are added as needed, followed by further centrifugation.

In the present invention, the concentration of PDGF-BB is preferably 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5 ng/mL.

In the present invention, the concentration of TGF-β is preferably 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50 ng/mL.

The composition of the present invention can be used as injectable or topical preparation, wherein a suitable skin matrix or injectable carrier, or an aqueous solution, non-aqueous solvent, suspension, emulsion or freeze-dried formulation, etc. may be used in the preparation, and be sterilized according to known methods.

The composition of the present invention may be combined with its pharmaceutically acceptable solvent, carrier or excipient to form a preparation. Examples of solvents include, but are not limited to, water, saline, sterile water, deionized water, glycerol, and so on.

The composition of the present invention may further contain additional raw materials, such as antimicrobials or preservatives.

### EMBODIMENTS

It should be understood that the detailed description of the embodiment is intended to illustrate the better embodiment of the present invention and not to limit the scope of the present invention.

### 1. Preparation of Enriched Platelet-Derived Growth Factor-BB (PDGF-BB) Growth Factor Dry Powder

The growth factor of the present invention is isolated from the whole blood of human or non-human mammals.

250 milliliters (mL) of whole blood was put into a blood bag containing an anticoagulant and centrifuged at 300˜1,500×g for 3 to 10 minutes to obtain platelet-rich plasma (PRP), also known as plasma layer solution.

Calcium chloride (CaCl₂)) activator was added to the PRP, centrifugation was performed again to remove the supernatant, so that the proteins of plasma were completely removed.

Same volume of water for injection as the removed supernatant was added, the growth factors released from activated platelet were obtained. These were then aliquoted into sample vials at 1 mL per vial. The vials were then freeze-dried to obtain the growth factor powder.

### 2-1. Preparation of Melanocyte-Specific Culture Medium Containing Enriched Platelet-Derived Growth Factor-BB (PDGF-BB) Dry Powder

1 mL of melanocyte-specific basal cell culture medium (including melanocyte-specific growth factor kit) was used to dissolve growth factor dry powder to prepare dry powder medium. Each gram of growth factor dry powder contains 15˜850 ng of PDGF-BB. The melanocyte-specific basal cell culture medium and dry powder medium were mixed in a certain proportion, then analyzed and evaluated by enzyme-linked immunosorbent assay (ELISA) to obtain enriched PDGF-BB dry powder culture medium with the final PDGF-BB concentration of 0 nanograms per milliliter (0 ng/ml), 0.12, 0.25, 0.5, 1.0, 2.0 ng/mL.

### 2-2. The Effect of Platelet-Derived Growth Factor-BB (PDGF-BB) on Melanocyte

The 96-well plate was divided into untreated wells (control group wells, 0 ng/ml PDGF-BB), and growth factor wells with 0.12, 0.25, 0.5, 1.0, and 2.0 ng/ml PDGF-BB, with 3 replicates per well.

Human Epidermal Melanocytes (HEM; Purchased from ATCC, product number PCS-200-012) were cultured in melanocyte-specific basal cell culture medium (purchased from ATCC, product number PCS-200-030) containing melanocyte-special growth factor kit (purchased from ATCC, product number PCS-200-041) and then replaced with fresh culture medium for later use.

HEM cells were added to untreated wells (control wells), 0.12, 0.25, 0.5, 1.0, and 2.0 ng/ml PDGF-BB growth factor wells, each well contained the same amounts of HEM cells, cells were cultured in an incubator at 37° C. and 5% carbon dioxide (CO₂) for 24 hours. Remove the old culture medium (used medium), enriched PDGF-BB dry powder culture medium corresponding to each group was added into the corresponding well and continued to incubate for 48 hours.

100 microliters (μL) of CCK-8 reagent (DOJINDO, CK-04-01) was added into the cells of the growth factor wells of 0 (control wells), 0.12, 0.25, 0.5, 1.0, and 2.0 ng/mL PDGF-BB, separately, placed in an incubator at 37° C. and 5% CO₂ for 1-3 hours. After the reaction was completed, the cell culture suspension from each well was aspirated, transferred into a microcentrifuge tube, and analyzed by using a multi-function microplate spectrophotometer (Varioskan LUX™, Thermo Scientific™) with absorbance value of 450 nm. The regression curve of absorbance value and cell number was established according to the CCK-8 reagent product manual, and the absorbance value was then converted to the number of viable cells.

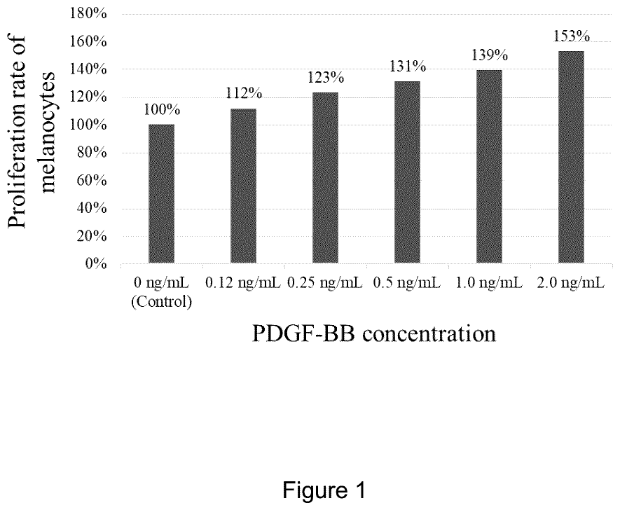

The experimental results are shown in table 1 and FIG. 1. The number of melanocytes in the control group is set as a benchmark. The results show that PDGF-BB dose-dependently promotes normal growth of melanocytes.

TABLE 1

| PDGF-BB | | | | | | |
|---|---|---|---|---|---|---|
| concentration | 0 | | | | | |
| (ng/mL) | (control) | 0.12 | 0.25 | 0.5 | 1.0 | 2.0 |
| Proliferation rate | 100% | 112% | 123% | 131% | 139% | 153% |
| of melanocytes | | | | | | |
| (%) | | | | | | |

### 2-3. The Effect of Platelet-Derived Growth Factor-BB (PDGF-BB) on Generation of Melanin of Melanocyte

The 96-well plate was divided into untreated wells (control group wells, 0 ng/ml PDGF-BB), and growth factor wells with 0.12, 0.25, 0.5, 1.0, and 2.0 ng/ml PDGF-BB, with 3 replicates per well.

Human Epidermal Melanocytes (HEM; Purchased from ATCC, product number PCS-200-012) were cultured in melanocyte-specific basal cell culture medium (purchased from ATCC, product number PCS-200-030) containing melanocyte-special growth factor kit (purchased from ATCC, product number PCS-200-041) and then replaced with fresh culture medium for later use.

HEM cells were added to untreated wells (control wells, 0 ng/ml PDGF-BB), 0.12, 0.25, 0.5, 1.0, and 2.0 ng/mL PDGF-BB growth factor wells, each well contained the same amounts of HEM cells, cells were cultured in an incubator at 37° C. and 5% carbon dioxide (CO₂) for 24 hours. Remove the old culture medium (used medium), enriched PDGF-BB dry powder culture medium corresponding to each group was added into the corresponding well and continued to incubate for 48 hours.

500 μL of TrypLE reagent (Gibco, A1285901) was added into the cells of the growth factor wells of 0 (control wells), 0.12, 0.25, 0.5, 1.0, and 2.0 ng/mL PDGF-BB, separately, placed in an incubator at 37° C. and 5% CO₂ for 3-5 minutes. After the reaction was completed, 500 μL of melanocyte-specific basal cell culture medium was added into each well to terminate the reaction of TrypLE reagent.

Cell fluid in each well was transferred into a microcentrifuge tube and centrifuged at 1,000-3,000 rpm (preferably, 2,000 rpm) for 10-30 minutes (preferably, 20 mins) with a microcentrifuge (Kubota, 3520) to remove the supernatant, and cell pellet was left. 110 μL of NaOH (Sigma, S5881) at a concentration of 1N was added into each tube for 10-20 minutes. After the reaction was completed, each mixture was transferred into a microcentrifuge tube and then analyzed using a multi-function microplate spectrophotometer (Varioskan LUX™, Thermo Scientific™) with absorbance value of 475 nm to obtain the absorbance of melanin.

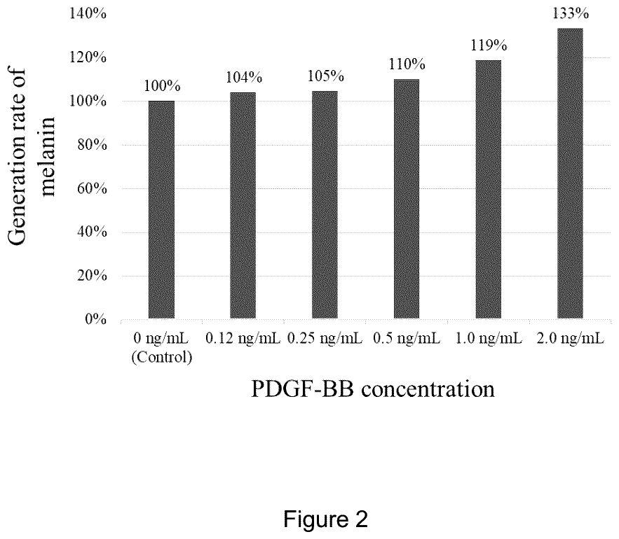

The experimental results are shown in table 2 and FIG. 2. The amount of melanin in the control group is set as a benchmark. The results show that melanin is increased simultaneously with the growth of melanocytes under the action of PDGF-BB.

TABLE 2

| PDGF-BB | | | | | | |
|---|---|---|---|---|---|---|
| concentration | 0 | | | | | |
| (ng/mL) | (control) | 0.12 | 0.25 | 0.5 | 1.0 | 2.0 |
| Generation rate | 100% | 104% | 105% | 110% | 119% | 133% |
| of melanin | | | | | | |
| (%) | | | | | | |

### 2-4. The Inhibition Effect of Platelet-Derived Growth Factor-BB (PDGF-BB) on Generation of Melanin

Based on the results of the above experiments, the inhibition rate of melanin generation per unit of melanocytes was calculated as follows:

$$Inhibition rate of melanin generation ( \% ) = ( 1 - the amount of melanin per unit of cell ( tested group ) ) \div the amount of melanin per unit of cell ( control group ) \times 100 \%$$

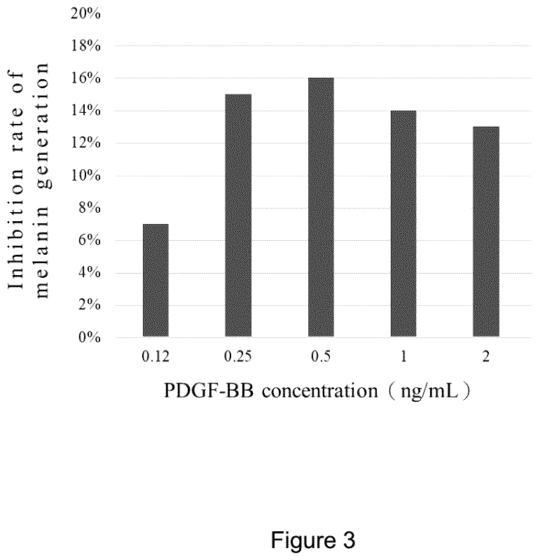

The experimental results are shown in table 3 and FIG. 3. The amount of melanin in the control group is set as a benchmark. After calculations, it was found that although melanin increased simultaneously with the growth of melanocytes under the action of PDGF-BB, PDGF-BB actually inhibited the growth of melanin in single melanocytes. When the PDGF-BB concentration reaches 0.12 ng/ml, the inhibition rate of melanin generation reaches 7%; when the PDGF-BB concentration reaches 0.25 ng/ml or more, the inhibition rate of melanin generation reaches 10% or more, and when the PDGF-BB concentration is in the range of 0.25 ng/ml to 1 ng/mL, the inhibition rate of melanin generation reaches 14% or even higher.

TABLE 3

| | | Inhibition rate of |
|---|---|---|
| PDGF-BB concentration | | melanin generation |
| (ng/mL) | Melanin/cell | (%) |
| 0 (control) | 1 | 0% |
| 0.12 | 0.93 | 7% |
| 0.25 | 0.85 | 15% |
| 0.5 | 0.84 | 16% |
| 1.0 | 0.86 | 14% |
| 2.0 | 0.87 | 13% |

### 3. Preparation of Enriched Transforming Growth Factor Beta (TGF-β) Growth Factor Dry Powder

The growth factor of the present invention is isolated from the whole blood of human or non-human mammals.

250 milliliters (mL) of whole blood was put into a blood bag containing an anticoagulant and centrifuged at 300˜1,500×g for 3 to 10 minutes to obtain platelet-rich plasma (PRP), also known as plasma layer solution.

Thrombin activator was added to the PRP, centrifugation was performed again to remove the supernatant, so that the proteins of plasma were completely removed.

Same volume of water for injection as the removed supernatant was added, the growth factors released from activated platelet were obtained. These were then aliquoted into sample vials at 1 mL per vial. The vials were then freeze-dried to obtain the growth factor powder.

### 4-1. Preparation of Melanocyte-Specific Culture Medium Containing Enriched Transforming Growth Factor Beta (TGF-β) Dry Powder

1 mL of melanocyte-specific basal cell culture medium (including melanocyte-specific growth factor kit) was used to dissolve growth factor dry powder to prepare dry powder medium. Each gram of growth factor dry powder contains 75˜8,500 ng of TGF-β. The melanocyte-specific basal cell culture medium and dry powder medium were mixed in a certain proportion, then analyzed and evaluated by enzyme-linked immunosorbent assay (ELISA) to obtain enriched TGF-β dry powder culture medium with the final TGF-β concentration of 0, 0.5, 1.0, 5.0, 10.0, 20.0 ng/mL.

### 4-2. The Inhibition Effect of TGF-β on Generation of Melanin

The 96-well plate was divided into untreated wells (control group wells, 0 ng/ml TGF-β), and growth factor wells with 0.5, 1.0, 5.0, 10.0, and 20.0 ng/ml TGF-β, with 3 replicates per well.

Human Epidermal Melanocytes (HEM; Purchased from ATCC, product number PCS-200-012) were cultured in melanocyte-specific basal cell culture medium (purchased from ATCC, product number PCS-200-030) containing melanocyte-special growth factor kit (purchased from ATCC, product number PCS-200-041) and then replaced with fresh culture medium for later use.

HEM cells were added to untreated wells (control wells, 0 ng/ml TGF-β), 0.5, 1.0, 5.0, 10.0, and 20.0 ng/ml TGF-β growth factor wells, each well contained the same amounts of HEM cells, cells were cultured in an incubator at 37° C. and 5% carbon dioxide (CO₂) for 24 hours. Remove the old culture medium (used medium), enriched TGF-β dry powder culture medium corresponding to each group was added into the corresponding well and continued to incubate for 48 hours.

500 μL of TrypLE reagent (Gibco, A1285901) was added into the cells of the growth factor wells of 0 (control wells), 0.5, 1.0, 5.0, 10.0, and 20.0 ng/ml TGF-β, separately, placed in an incubator at 37° C. and 5% CO₂ for 3-5 minutes. After the reaction was completed, 500 μL of melanocyte-specific basal cell culture medium was added into each well to terminate the reaction of TrypLE reagent.

Cell fluid in each well was transferred into a microcentrifuge tube and centrifuged at 1,000-3,000 rpm (preferably, 2,000 rpm) for 10-30 minutes (preferably, 20 mins) with a microcentrifuge (Kubota, 3520) to remove the supernatant, and cell pellet was left. 110 μL of NaOH (Sigma, S5881) at a concentration of 1N was added into each tube for 10-20 minutes. After the reaction was completed, each mixture was transferred into a microcentrifuge tube and then analyzed using a multi-function microplate spectrophotometer (Varioskan LUX™ Thermo Scientific™) with absorbance value of 475 nm to obtain the absorbance of melanin.

The inhibition rate of melanin generation per unit of melanocytes was calculated as follows:

$$Inhibition rate of melanin generation ( \% ) = ( 1 - the amount of melanin per unit of cell ( tested group ) ) \div the amount of melanin per unit of cell ( control group ) \times 100 \%$$

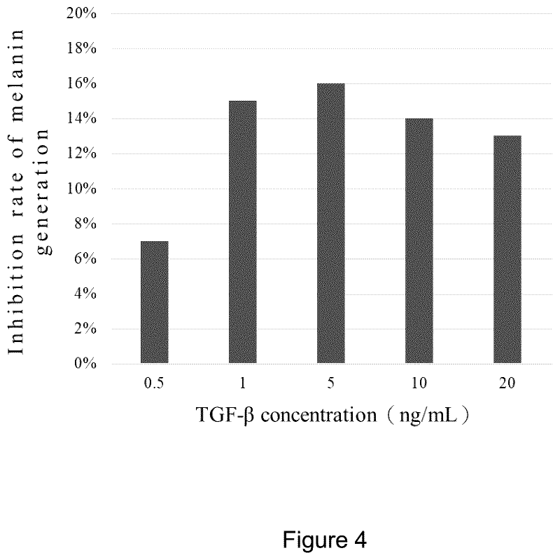

The experimental results are shown in table 4 and FIG. 4. The amount of melanin in the control group is set as a benchmark. After calculations, when the TGF-β concentration reaches 0.5 ng/ml, the inhibition rate of melanin generation reaches 7%; when the TGF-β concentration reaches 1.0 ng/ml or more, the inhibition rate of melanin generation reaches 10% or more, and when the TGF-β concentration is in the range of 1.0 ng/mL to 10 ng/ml, the inhibition rate of melanin generation reaches 14% or even higher.

TABLE 4

| | | Inhibition rate of |
|---|---|---|
| TGF-β concentration | | melanin generation |
| (ng/mL) | Melanin/cell | (%) |
| 0 (control) | 1 | 0% |
| 0.5 | 0.93 | 7% |
| 1.0 | 0.85 | 15% |
| 5.0 | 0.84 | 16% |
| 10.0 | 0.86 | 14% |
| 20.0 | 0.87 | 13% |

### 5. Preparation of Platelet Dry Powder

The platelet of the present invention is isolated from the whole blood of human or non-human mammals.

250 milliliters (mL) of whole blood was put into a blood bag containing an anticoagulant and centrifuged at 300˜1,500×g for 3 to 10 minutes to obtain platelet-rich plasma (PRP), also known as plasma layer solution.

Centrifugation was performed again until platelets have collected at the bottom, removed the supernatant, so that the proteins of plasma were completely removed. Same volume of water for injection as the removed supernatant was added, and the purified platelets were obtained. These were then aliquoted into sample vials at 1 mL per vial. The vials were then freeze-dried to obtain the platelet powder.

### 6-1. Preparation of Platelet Culture Medium

1 mL of melanocyte-specific basal cell culture medium (including melanocyte-specific growth factor kit) was used to dissolve platelet dry powder to prepare platelet dry powder medium. Each gram of growth factor dry powder contains 4×10⁸˜1.5×10¹¹ platelets. The melanocyte-specific basal cell culture medium and platelet dry powder medium were mixed in a certain proportion to prepare melanocyte-specific culture medium containing different concentrations of platelet, namely: 0 platelet (control group), 1.25×10⁷ platelets, 2.5×10⁷ platelets, 5×10⁷ platelets, 10×10⁷ platelets, and 15×10⁷ platelets.

### 6-2. The Inhibition Effect of Platelet on Generation of Melanin

The effect of the amount of platelet on melanin was further investigated in the present invention.

The 96-well plate was divided into untreated wells (control group wells, 0 platelet), 1.25×10⁷ platelets, 2.5×10⁷ platelets, 5×10⁷ platelets, 10×10⁷ platelets, and 15×10⁷ platelets, with 3 replicates per well.

HEM cells were added to untreated wells (control wells), treated wells with 1.25×10⁷ platelets, 2.5×10⁷ platelets, 5×10⁷ platelets, 10×10⁷ platelets, and 15×10⁷ platelets, each well contained the same amounts of HEM cells, cells were cultured in an incubator at 37° C. and 5% carbon dioxide (CO₂) for 24 hours. Remove the old culture medium (used medium), platelet dry powder culture medium corresponding to each group was added into the corresponding well and continued to incubate for 48 hours.

500 μL of TrypLE reagent (Gibco, A1285901) was added into each well cells, placed in an incubator at 37° C. and 5% CO₂ for 3-5 minutes. After the reaction was completed, 500 μL of melanocyte-specific basal cell culture medium was added into each well to terminate the reaction of TrypLE reagent.

Cell fluid in each well was transferred into a microcentrifuge tube and centrifuged at 1,000-3,000 rpm (preferably, 2,000 rpm) for 10-30 minutes (preferably, 20 mins) with a microcentrifuge (Kubota, 3520) to remove the supernatant, and cell pellet was left. 110 μL of NaOH (Sigma, S5881) at a concentration of 1N was added into each tube for 10-20 minutes. After the reaction was completed, each mixture was transferred into a microcentrifuge tube and then analyzed using a multi-function microplate spectrophotometer (Varioskan LUX™ Thermo Scientific™) with absorbance value of 475 nm to obtain the absorbance of melanin.

The inhibition rate of melanin generation per unit of melanocytes was calculated as follows:

$$Inhibition rate of melanin generation ( \% ) = ( 1 - the amount of melanin per unit of cell ( tested group ) ) \div the amount of melanin per unit of cell ( control group ) \times 100 \%$$

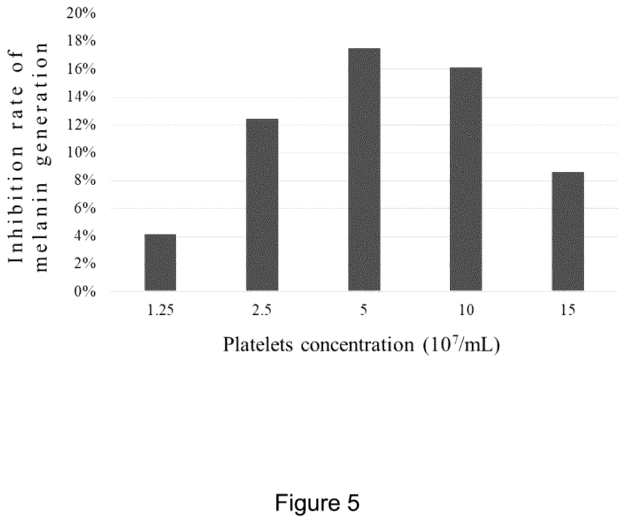

The experimental results are shown in table 5 and FIG. 5. The amount of melanin in the control group is set as a benchmark. After calculations, when the platelet concentration is in the range of 2.5×10⁷ to 15×10⁷, the inhibition rate of melanin generation reaches 8% or more; When the platelet concentration is in the range of 2.5×10⁷ to 10×10⁷, the inhibition rate of melanin generation is as high as 12% or more, and when the platelet concentration is in the range of 5×10⁷ to 10×10⁷, the inhibition rate of melanin generation is as high as 16% or more.

TABLE 5

| | | | | Inhibition rate |
|---|---|---|---|---|
| | The | The | | of melanin |
| | number of | amounts of | | generation |
| Group | melanocytes | melanin | Melanin/Cell | (%) |
| Control | 177,221 | 0.05515 | 3.11 × 10⁷ | 0% |
| 1.25 × 10⁷ | 209,321 | 0.0625 | 2.99 × 10⁷ | 4.10% |
| 2.5 × 10⁷ | 230,078 | 0.0627 | 2.73 × 10⁷ | 12.40% |
| 5 × 10⁷ | 268,700 | 0.069 | 2.57 × 10⁷ | 17.50% |
| 10 × 10⁷ | 294,707 | 0.07695 | 2.61 × 10⁷ | 16.10% |
| 15 × 10⁷ | 318,921 | 0.09075 | 2.85 × 10⁷ | 8.60% |

### 7-1. Preparation of Platelet Culture Medium

1 mL of melanocyte-specific basal cell culture medium (including melanocyte-specific growth factor kit) was used to dissolve platelet dry powder to prepare platelet dry powder medium. The melanocyte-specific basal cell culture medium and platelet dry powder medium were mixed in a certain proportion to prepare melanocyte-specific culture medium containing different concentrations of platelet, namely: 0 platelet (control group), 1.25×10⁷ platelets, 2.5×10⁷ platelets, 5×10⁷ platelets, and 10×10⁷ platelets.

### 7-2. Preparation of Melanocyte-Specific Culture Medium Containing Different Whitening Ingredients

Arbutin (Sigma, SI-A4256) was prepared with melanocyte-specific basal cell culture medium to achieve a final concentration of 50 μg/mL of arbutin. Glutathione (Sigma, SI-G6013) was prepared with melanocyte-specific basal cell culture medium to achieve a final concentration of 100 μg/mL of glutathione. Tranexamic acid (Siuguan, Taiwan FDA Pharmaceutical Manufacturing License No. 027356) was prepared with melanocyte-specific basal cell culture medium, so that the final concentration of Tranexamic acid was 2 mg/mL.

### 7-3. The Effect of Platelets and Different Whitening Ingredients on Inflammation of Melanocytes

The 96-well plate was divided into control group wells (0 platelet), treated wells containing 1.25×10⁷ platelets, 2.5×10⁷ platelets, 5×10⁷ platelets, and 10×10⁷ platelets, Arbutin wells, Glutathione wells, and Tranexamic acid wells, with 3 replicates per well.

HEM cells were added into each well, separately, and each well contained the same amounts of HEM cells, cells were cultured in an incubator at 37° C. and 5% carbon dioxide (CO₂) for 24 hours. Remove the old culture medium (used medium), melanocyte-specific culture medium prepared in steps 6-1 and 6-2 corresponding to each group was added into the corresponding well and continued to incubate for 48 hours.

The cell culture suspension in each well was transferred into a microcentrifuge tube. The concentration of Tumor Necrosis Factor-α (TNF-α) contained in the cell culture suspension was detected by using human TNF-α ELISA kit (abcam, ab181421) and analyzed by using a multi-function microplate spectrophotometer (Varioskan LUX™, Thermo Scientific™) with absorbance value of 450 nm. The regression curve of absorbance value and TNF-α was established according to the TNF-α ELISA kit product manual, and the absorbance value was then converted into TNF-α concentration to evaluate the inhibition effect of inflammation of melanocytes. The inhibition rate was calculated as follows:

$$Inhibition rate of inflammation ( \% ) = 1 - ( TNF - α concentration ( tested group ) \div TNF - α concentration ( control group ) ) \times 100 \%$$

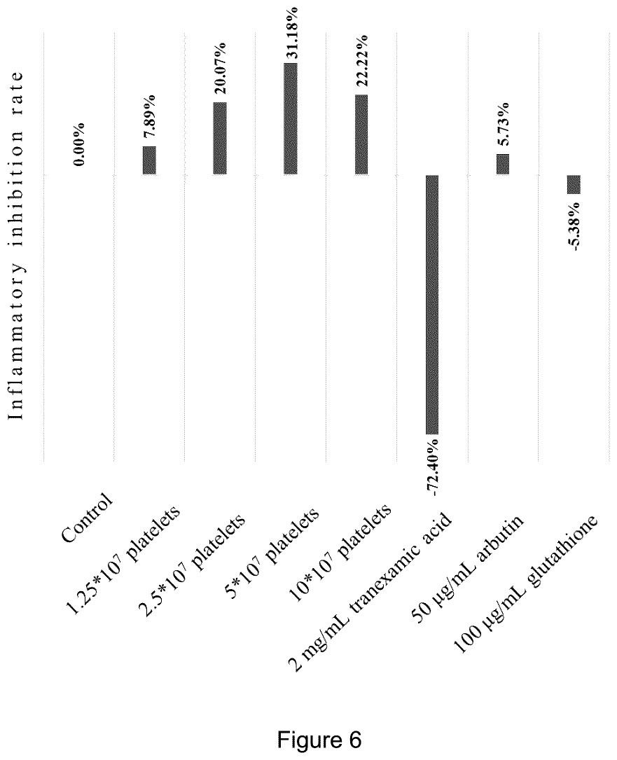

The experimental results are shown in table 6 and FIG. 6. The TNF-α concentration of control group is set as a benchmark. After calculations, it was found that each platelet group has the ability to alleviate inflammation and is significantly better than common whitening ingredients on the market.

TABLE 6

| | TNF-α | Inflammatory inhibition |
|---|---|---|
| Group | (pg/mL) | rate (%) |
| Control | 27.9 | 0% |
| 1.25 × 10⁷ platelets | 25.7 | 7.89% |
| 2.5 × 10⁷ platelets | 22.3 | 20.07% |
| 5 × 10⁷ platelets | 19.2 | 31.18% |
| 10 × 10⁷ platelets | 21.7 | 22.22% |
| 2 mg/mL tranexamic acid | 48.1 | −72.40% |
| 50 μg/mL arbutin | 26.3 | 5.73% |
| 100 μg/mL glutathione | 29.4 | −5.38% |

### 8-1. Preparation of Platelet Culture Medium

1 mL of melanocyte-specific basal cell culture medium (including melanocyte-specific growth factor kit) was used to dissolve platelet dry powder to prepare platelet dry powder medium. The melanocyte-specific basal cell culture medium and platelet dry powder medium were mixed in a certain proportion to prepare melanocyte-specific culture medium containing different concentrations of platelet, namely: 0 platelet (control group), 1.25×10⁷ platelets, 2.5×10⁷ platelets, 5×10⁷ platelets, and 10×10⁷ platelets.

### 8-2. Preparation of Melanocyte-Specific Culture Medium Containing Different Whitening Ingredients

Arbutin (Sigma, SI-A4256) was prepared with melanocyte-specific basal cell culture medium to achieve a final concentration of 50 μg/mL of arbutin. Glutathione (Sigma, SI-G6013) was prepared with melanocyte-specific basal cell culture medium to achieve a final concentration of 100 μg/mL of glutathione. Tranexamic acid (Siuguan, Taiwan FDA Pharmaceutical Manufacturing License No. 027356) was prepared with melanocyte-specific basal cell culture medium, so that the final concentration of Tranexamic acid was 2 mg/mL.

### 8-3. The Effect of Platelets and Different Whitening Ingredients on Tyrosinase Activity

The 96-well plate was divided into control group wells (0 platelet), treated wells containing 1.25×10⁷ platelets, 2.5×10⁷ platelets, 5×10⁷ platelets, and 10×10⁷ platelets, Arbutin wells, Glutathione wells, and Tranexamic acid wells, with 3 replicates per well.

HEM cells were added into each well, separately, and each well contained the same amounts of HEM cells, cells were cultured in an incubator at 37° C. and 5% carbon dioxide (CO₂) for 24 hours. Remove the old culture medium (used medium), melanocyte-specific culture medium prepared in steps 7-1 and 7-2 corresponding to each group was added into the corresponding well and continued to incubate for 48 hours.

The cell culture suspension in each well was transferred into a microcentrifuge tube. The concentration of tyrosinase contained in the cell culture suspension was detected by using human tyrosinase ELISA kit (antibodies, A312872) and analyzed by using a multi-function microplate spectrophotometer (Varioskan LUX™, Thermo Scientific™) with absorbance value of 450 nm. The regression curve of absorbance value and tyrosinase was established according to the tyrosinase ELISA kit product manual, and the absorbance value was then converted into tyrosinase concentration to evaluate the inhibition effect of tyrosinase activity. The inhibition rate was calculated as follows:

$$Inhibition rate of tyrosinase ( \% ) = 1 - ( tyrosinase concentration ( tested group ) \div tyrosinase concentration ( control group ) ) \times 100 \%$$

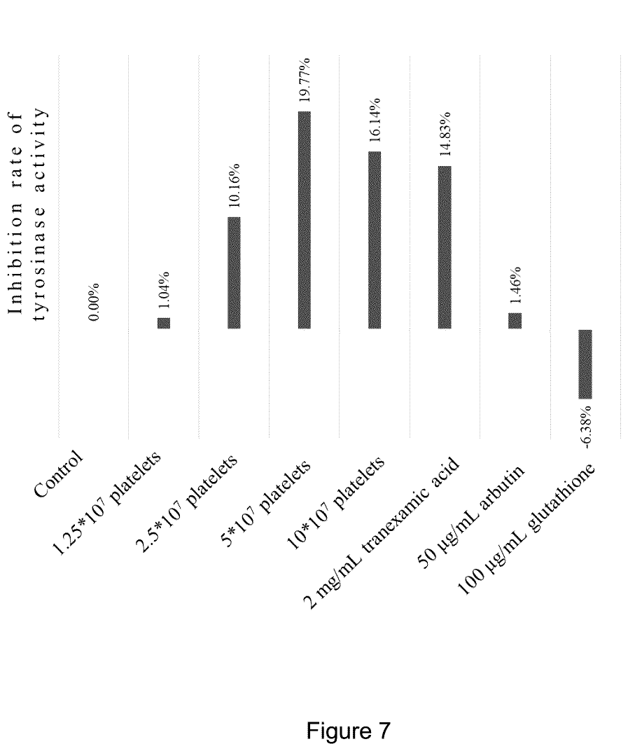

The experimental results are shown in table 7 and FIG. 7. The tyrosinase concentration of control group is set as a benchmark for the calculation of inhibition rate of tyrosinase activity. After calculations, it was found that each platelet group has the ability to inhibit tyrosinase activity and is significantly better than common whitening ingredients on the market.

TABLE 7

| | Tyrosinase | Inhibition rate of |
|---|---|---|
| Group | (pg/mL) | tyrosinase activity (%) |
| Control | 327.8 | 0% |
| 1.25 × 10⁷ platelets | 324.4 | 1.04% |
| 2.5 × 10⁷ platelets | 294.5 | 10.16% |
| 5 × 10⁷ platelets | 263.0 | 19.77% |
| 10 × 10⁷ platelets | 274.9 | 16.14% |
| 2 mg/mL tranexamic acid | 279.2 | 14.83% |
| 50 μg/mL arbutin | 323.0 | 1.46% |
| 100 μg/mL glutathione | 348.7 | −6.38% |

What is claimed is:

1. A composition for inhibiting generation of melanin, comprising platelets and/or growth factors isolated from blood.

2. The composition of claim 1, wherein the composition comprises platelets isolated from plasma.

3. The composition of claim 2, wherein the composition comprises 2.5×10⁷ to 10×10⁹ platelets.

4. The composition of claim 3, wherein the composition comprises 2.5×10⁷ to 10×10⁸ platelets.

5. The composition of claim 2, wherein the composition comprises 5×10⁷ to 10×10⁸ platelets.

6. The composition of claim 1, wherein the growth factors is selected from platelet-derived growth factor-BB (PDGF-BB) or transforming growth factor (TGF-β).

7. The composition of claim 6, wherein the concentration of PDGF-BB is 0.1 ng/ml to 5 ng/mL.

8. The composition of claim 7, wherein the concentration of PDGF-BB is 0.1 ng/ml to 3 ng/mL.

9. The composition of claim 8, wherein the concentration of PDGF-BB is 0.25 ng/ml to 2 ng/mL

10. The composition of claim 6, wherein the concentration of TGF-β is 0.5 ng/mL to 50 ng/mL.

11. The composition of claim 10, wherein the concentration of TGF-β is 0.5 ng/ml to 30 ng/mL.

12. The composition of claim 11, wherein the concentration of TGF-β is 1 ng/ml to 20 ng/mL

13. The composition of claim 1, wherein the composition is a dry powder prepared by freeze drying.

14. The composition of claim 13, wherein the dry powder comprises 4×10⁸˜1.5×10¹¹ platelets per gram.

15. The composition of claim 13, wherein the dry powder comprises 15 to 850 ng of PDGF-BB per gram.

16. The composition of claim 13, wherein the dry powder comprises 75 to 8,500 ng of TGF-β per gram.

17. A method for alleviating skin pigmentation, comprising administering a composition of claim 1 to a subject suffering from skin pigmentation.

18. The method of claim 17, wherein the skin pigmentation comprises melasma.
